(19) United States
(12) Patent Application Publication
(10) Pub. No.: US 2023/0307088 A1
(43) Pub. Date: Sep. 28, 2023

(54) Deep Learning for De Novo Antibody Affinity Maturation (Modification) and Property Improvement

(71) Applicant: Flagship Pioneering Innovations VI, LLC, Cambridge, MA (US)

(72) Inventors: Zachary Kohl Costello, Cambridge, MA (US); Jacob Feala, Franklin, MA (US); Andrew Lane Beam, Jamaica Plain, MA (US)

(21) Appl. No.: 18/006,788

(22) PCT Filed: Jul. 28, 2021
(86) PCT No.: PCT/US2021/043461

§ 371 Date: Jan. 25, 2023

Related U.S. Application Data
(60) Provisional application No. 63/057,376, filed on Jul. 28, 2020.

(51) Int. Cl. G16B 15/30; G16B 15/20; G16B 40/20; G06N 3/088

(52) U.S. Cl. CPC G16B 15/30; G16B 15/20; G16B 40/20; G06N 3/088

(57) ABSTRACT

Controlling antibody affinity and expression are key to clinical applications. High affinity antibodies correlate with higher specificity and can be used at lower doses. Presently, antibody maturation is tackled with directed evolution methods. In this case, an initial library of mutated binders is seeded into a process and affinity is improved through multiple rounds of mutation and selection. However, the present disclosure employs a machine learning approach to computationally mature antibody sequences using a process having parallels to directed evolution. These antibody sequences can be manufactured into physical antibodies after their computation and verification. Additionally, the present method has the potential to outperform directed evolution when targeting a specific affinity, and is applicable to general protein-protein interactions.

### RELATED APPLICATION(S)

This application claims the benefit of U.S. Provisional Application No. 63/057,376, filed on Jul. 28, 2020. The entire teachings of the above applications are incorporated herein by reference.

### BACKGROUND

Antibody maturation is the process of improving an affinity of a given antibody to its antigen. In immunology, an antigen is a toxin or foreign substance that induces an immune response. An example of such an immune response is the production of antibodies that bind to antigens to counteract the antigen. Antibodies can also be designed by a variety of methods.

### SUMMARY

Controlling antibody affinity and expression are key to clinical applications. High affinity antibodies correlate with higher specificity and can be used at lower doses. Presently, antibody maturation is tackled with directed evolution methods. In this case, an initial library of mutated binders is seeded into a process and affinity is improved through multiple rounds of mutation and selection. However, the present disclosure employs a machine learning approach to computationally mature antibody sequences using a process having parallels to directed evolution. These antibody sequences can be manufactured into physical antibodies after their computation and verification. Additionally, the present method has the potential to outperform directed evolution when targeting a specific affinity, and is applicable to general protein-protein interactions.

In an embodiment, computational directed evolution (CDE) can be used in multiobjective and multimodel situations, e.g., when selecting for improvements in two or more properties substantially simultaneously. In this case, one or more models can be used. Each of these models may have one or more property to be optimized. The one or more models can be combined into a single objective function, and CDE can optimize that objective. The objective function is optimized with an optimization procedure (in this case, computational directed evolution). When the objective function is optimized, it results in antibody sequences that improve upon of one or more antibody properties of interest.

In an embodiment, a method for determining an antibody sequence having an improved property includes training a machine learning model based on a first group of antibody sequences. Each antibody sequence of the database is labeled with one or more corresponding properties. The method further generates a fine-tuned machine learning model by training the machine learning model based on a second plurality of antibody sequences, each of the second plurality of antibody sequences labeled with a corresponding property. The second plurality of antibody sequences is related to an antigen of interest. The method further includes generating an antibody sequence based on the fine-tuned machine learning model.

A person of ordinary skill in the art is that a fine-tuned machine learning model (also a finely tuned machine learning model) is a machine learning model that is first trained on a general dataset, and then finely tuned on a more specific dataset. Fine-tuning can also be described as a process to take a machine learning model that has been trained for a given task, and training it to perform a second task.

In an embodiment, the method for determining an antibody sequence having an improved property includes generating a score for each a plurality of machine learning models. Each machine learning model is trained based on a respective plurality of antibody sequences. Each antibody sequence of the respective plurality of antibody sequences is labeled with a property corresponding to the plurality of antibody sequences and a value of the property corresponding to the respective antibody sequence. Generating the score for each machine learning model results in a respective score for each machine learning model of the plurality of machine learning models indicating a contribution to predicting the property corresponding to the respective machine learning model. The method further includes generating an antibody sequence using the plurality of machine learning models by weighting outputs of each machine learning model according to each score generated and combining the weighted outputs into a weighted sum.

In embodiments, the contribution includes one or more of an importance of the property to manufacturing, an expression of a protein, an immunogenicity in a patient, expression, developability, an interaction with other models, orthogonality with other models, and an empirical derivation by tuning the generation process.

In an embodiment, generating the antibody sequence further includes selecting an antibody sequence from a proposal distribution based on the fine-tuned machine learning model. Generating the antibody sequence further includes determining whether the antibody sequence selected has a probability of acceptance over a particular threshold, and if so, analyzing the antibody, and if not, selecting a next antibody sequence from the proposal distribution.

In an embodiment, generating the antibody sequence further includes comparing a first property value, determined by the fine-tuned machine learning model, of an antibody selected from a proposal distribution to a second property value, determined by the fine-tuned machine learning model, of an antibody having a best property value in a current search. If the first property value is greater than the second property value, the method replaces the antibody having the best property value with the antibody selected from the proposal distribution.

In an embodiment, training the machine learning model further includes providing a set of amino acid sequences labeled with at least one property. Training the machine learning model further includes masking a portion of the set of amino acid sequences to provide a masked set of amino acid sequences. The remainder of the set of amino acid sequences is an unmasked set of amino acid sequences. Training the machine learning model further includes training the machine learning model to estimate each of the masked set of amino acid sequences based on (1) the at least one property labeling each masked amino acid sequence and (2) the unmasked set of amino acid sequences and the labeled properties of each unmasked amino acid sequence.

In an embodiment, generating the fine-tuned machine learning model further includes weighting each sequence property of the second plurality of antibody sequences. Generating the fine-tuned machine learning model further includes determining optimum model parameters to generate the second plurality of antibody sequences using the machine learning model. Generating the fine-tuned machine learning model further includes applying the optimum model parameters to the machine learning model. The resulting model having the optimum model parameters applied being the fine-tuned machine learning model.

In an embodiment, the corresponding property is affinity (e.g., binding affinity) or expression. In other embodiments, examples of properties (e.g., the function values) can be one or more of: binding affinity, binding specificity, catalytic (e.g., enzymatic) activity, fluorescence, solubility, thermal stability, conformation, immunogenicity, protein aggregation, proteolytic stability, expression, off target effects, and any other functional property of biopolymer sequences. This process is applicable to any proteins where we have a (small) starting set of examples which have the property of interest. Then from there we can use the process described here to modify the property of interest to be better suited to our needs. This could be in increase in the value (e.g., an increase in catalytic rate), a decrease in property value (e.g., a decrease in immunogenicity) or to hit a target value or range of values (e.g., hit a specific binding affinity).

In an embodiment, the method includes selecting an antibody sequence candidate from a proposal distribution based on the fine-tuned machine learning model, the antibody sequence being within a defined acceptance criterion. The method further includes, if the property of the antibody sequence candidate is greater than a best found antibody sequence, replacing the best found antibody sequence with the antibody sequence candidate, or otherwise, disregarding the antibody sequence candidate.

In an embodiment, the method also includes producing an antibody having the antibody sequence generated. In an embodiment, the method can also include providing a manufactured antibody having the antibody sequence generated and assaying the antibody for the property.

In an embodiment, a system for determining an antibody having an improved property includes a processor and a memory with computer code instructions stored thereon. The processor and the memory, with the computer code instructions, are configured to cause the system to train a machine learning model based on a first plurality of antibody sequences. Each antibody sequence of the database being labeled with a corresponding property. The processor is further configured to generate a fine-tuned machine learning model by training the machine learning model based on a second plurality of antibody sequences and corresponding properties. The second plurality of antibody sequences related to an antigen of interest. The processor is further configured to generate an antibody sequence based on the fine-tuned machine learning model.

In an embodiment, a method of antibody maturation can include providing a first antibody sequence to the system or methods described above, and obtaining, from the system, the generated antibody sequence.

In an embodiment, an isolated antibody can be produced by the above method. In an embodiment, the isolated antibody is recombinantly produced. In an embodiment, the isolated antibody is chemically synthesized.

In an embodiment, a method includes determining or generating an antibody sequence having an improved property, the determining or generating being performed by a fine-tuned machine learning model. The fine-tuned machine learning model can be generated by (1) training a machine learning model based on a first plurality of antibody sequences, each antibody sequence of the first plurality of antibody sequences being labeled with a corresponding property, and (2) generating the fine-tuned machine learning model by training the machine learning model based on a second plurality of antibody sequences, each antibody sequence of the second plurality of antibody sequences being labeled with a corresponding property, the second plurality of antibody sequences related to an antigen of interest. Optionally, training the machine learning model, generating the fine-tuned machine learning model, or both can be performed by a third party separate from generating the antibody sequence.

As used in this specification, antibody sequence refers to an ordered sequence of amino acids that can be stored digitally or in another format. An antibody refers to a physical manifestation of an antibody. A person having ordinary skill in the art can recognize that an antibody sequence generated by the systems and methods of this disclosure can be manufactured or otherwise produced as an antibody.

In an embodiment, method for determining an antibody sequence having an improved property includes training a plurality of machine learning models. Each machine learning model is trained based on a corresponding initial plurality of antibody sequences. Each antibody sequence of the initial plurality of antibody sequences is labeled with a corresponding property. A person of ordinary skill in the art can understand that each respective machine learning model may be trained by a different plurality of antibody sequences. The method then generates a plurality of fine-tuned machine learning models. Each fine-tune machine learning model is generated by training each machine learning model with a secondary plurality of antibody sequences. Each antibody sequence of the secondary plurality of antibody sequences are labeled with a corresponding property, and the secondary plurality of antibody sequences is related to an antigen of interest. The method then generates an antibody sequence based an objective function using the plurality of fine-tuned machine learning models weighted by corresponding hyperparameters.

In an embodiment, one or more of the antibody sequences is related to an antigen of interest.

In an embodiment, a method for determining an antibody sequence having an improved property includes providing a score for each of a plurality of machine learning models, each machine learning model trained based on a respective plurality of antibody sequences. Each antibody sequence of the respective plurality of antibody sequences is labeled with a property corresponding to the plurality of antibody sequences and a value of the property corresponding to the respective antibody sequence. Each respective score for each machine learning model of the plurality of machine learning models indicates a contribution to predicting the property corresponding to the respective machine learning model. The method further includes generating an antibody sequence using the plurality of machine learning models by weighting outputs of each machine learning model according to each score provided and combining the weighted outputs into a weighted sum.

In an embodiment, a method for determining an antibody sequence having an improved property includes generating an antibody sequence using a plurality of machine learning models. The generating of the antibody sequence is performed by weighting outputs of each machine learning model according to a score for each of the plurality of machine learning models. Each machine learning model is trained based on a respective plurality of antibody sequences. Each antibody sequence of the respective plurality of antibody sequences labeled with a property corresponding to the plurality of antibody sequences and a value of the property corresponding to the respective antibody sequence. Each respective score for each machine learning model of the plurality of machine learning models indicating a contribution to predicting the property corresponding to the respective machine learning model. The method further includes combining the weighted outputs into a weighted sum.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a block diagram illustrating an example embodiment of a method of the present disclosure.

FIG. 1B is a flow diagram illustrating an example embodiment of a process employed by the present disclosure.

FIG. 2 is a graph illustrating a random walk sequence in an antibody sequence space for discovering antibody sequences using Computational Directed Evolution.

FIG. 3 is a graph illustrating the improvements of the generated antibody sequences compared to existing datasets.

FIG. 4 illustrates a computer network or similar digital processing environment in which embodiments of the present invention may be implemented.

FIG. 5 is a diagram of an example internal structure of a computer (e.g., client processor/device or server computers) in the computer system of FIG. 4.

### DETAILED DESCRIPTION

A description of example embodiments follows.

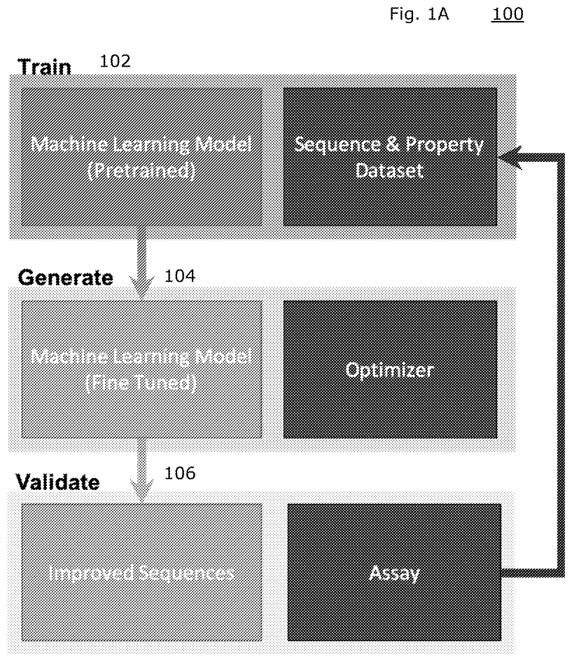

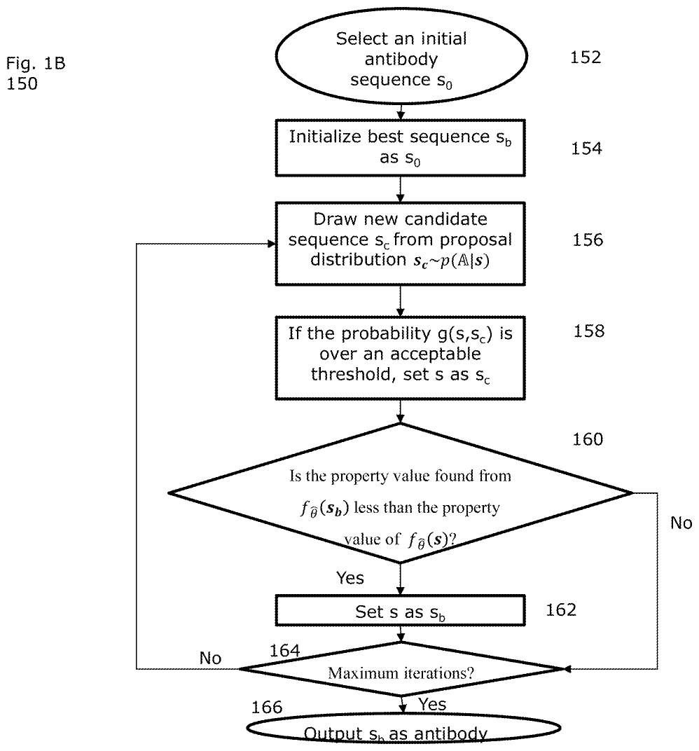

FIG. 1A is a block diagram 100 illustrating an example embodiment of a method of the present disclosure. The present disclosure takes a de novo approach to closed loop antibody affinity maturation. The iterative process begins with unsupervised pretraining of a deep machine learning model on a large and relevant set of protein sequence data and their properties (102). In embodiments, this includes unsupervised pretraining of a language model on a dataset containing n antibody sequences. This conditions the model to learn the underlying statistical relationships between amino acids in antibody sequences.

The process then fine-tunes the pretrained model on a smaller supervised learning task using data specific to the desired antibody antigen pair (104). This fine-tuned machine learning model is trained jointly on both affinity and expression as the data specific to the desired antibody antigen pair, though in practice any number or type of additional properties can be used in the finetuning process. Once the finetuned model is trained (104), the model is employed downstream to perform affinity maturation by solving an optimization problem described further below. After finetuning, computational directed evolution performs a constrained search of antibody sequence space for high affinity antibody sequences to a chosen target. The solution to this optimization problem is one or more affinity matured antibody sequences (e.g., sequences that are predicted by the machine learning model to have a higher affinity than observed in the supervised training task) (106). The method then constructs these affinity matured antibody sequences (or improved antibody sequences). The method further experimentally assays the constructed antibody sequences for affinity and expression. The new data from the assayed sequences is incorporated into the supervised learning dataset, and the process 100 repeats until suitable candidates are found (102). In an embodiment, a qualification for a high affinity candidate can be an equilibrium dissociation constant (K_{D}) of less than 10 picomolar (pM) (for example, in the case of antibodies against fluorescein).

As used herein, “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv), single chain variable fragment (scFv), mutants thereof, fusion proteins comprising an antibody portion, humanized antibodies, chimeric antibodies, diabodies, linear antibodies, single chain antibodies, single domain antibodies (e.g., camel or llama VHH antibodies), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

As used herein the term “KID,” also referred to as “binding constant,” “equilibrium dissociation constant” or “affinity constant,” is a measure of the extent of a reversible association between two molecular species (e.g., antibody and target protein) and includes both the actual binding affinity as well as the apparent binding affinity. Binding affinity can be determined using methods known in the art including, for example, by measurement of surface plasmon resonance, e.g., using a BIAcore system and assay.

In some aspects, the antibody binds a target protein with a K_{D} of less than 10⁻⁴ M, 10⁻⁵M, 10⁻⁶M, 10⁻⁷M, 10⁻⁸M, 10⁻⁹M, 10⁻¹⁰ M, 10⁻¹¹ M, or 10⁻¹² M. For example, the antibody can bind a target protein with a K_{D} of less than 1000 nM, or alternatively less than 900 nM, or alternatively less than 800 nM, or alternatively less than 700 nM, or alternatively less than 600 nM, or alternatively less than 500 nM, or alternatively less than 400 nM, or alternatively less than 300 nM, or alternatively less than 200 nM, or alternatively less than 100 nM, or alternatively less than 90 nM, or alternatively less than 80 nM, or alternatively less than 70 nM, or alternatively less than 60 nM, or alternatively less than 50 nM, or alternatively less than 40 nM, or alternatively less than 30 nM, or alternatively less than 20 nM, or alternatively less than 15 nM, or alternatively less than 10 nM, or alternatively less than 9 nM, or alternatively less than 8 nM, or alternatively less than 7 nM, or alternatively less than 6 nM, or alternatively less than 5 nM, or alternatively less than 4 nM, or alternatively less than 3 nM, or alternatively less than 2 nM, or alternatively less than 1 nM, or alternatively less than 1000 pM, or alternatively less than 100 pM, or or alternatively less than 10 pM, or alternatively less than 1 pM.

In some aspects, an antibody sequence identified by the methods disclosed herein correspond to an antibody that binds a target protein with at least 70%, or alternatively at least 75%, or alternatively at least 80%, or alternatively at least 85%, or alternatively at least 90%, or alternatively at least 95% affinity or higher affinity compared to the affinity of a reference antibody. In certain aspects, the antibody binds a target protein with higher affinity than a reference antibody. The reference antibody can be, for example, an antibody having the highest reported (e.g., published) affinity for a given target.

In practice, the process 100 described in FIG. 1A rapidly improves antibody affinity after one round, with the generation of fluorescein antibody sequences that have greater than 10 pM affinity. This process can reduce the cost and time it takes to generate better therapeutic antibodies by reducing the number of constructs that need to be made and tested. The details of this process are described below.

In an initial experiment, the process employed two datasets: a large unsupervised set of antibody sequences, and a small set of sequence affinity pairs for the antibody of interest to computationally bootstrap maturation. Specifically, pretraining used the complete antibody repertoire taken from a single human. This dataset is described in further detail in Bryan, et al., “Commonality despite exceptional diversity in the baseline human antibody repertoire,” Nature 566.7744 (2019): 393 (hereinafter “Bryan”), which is hereby incorporated by reference in its entirety. The unsupervised dataset, U, contains approximately 300 million antibody sequences, s E U. For finetuning, high throughput fluorescein antibody data is used from Adams, Rhys M., et al. “Measuring the sequence-affinity landscape of antibodies with massively parallel titration curves.” Elife 5 (2016): e23156 (hereinafter “Adams”), which is hereby incorporated by reference in its entirety. This example supervised dataset, 5, is comprised of 2803 sequences paired with expression and affinity data evaluated in triplicate. Examples from this dataset (s,a,e)∈ [symbol] , are tuples containing a protein sequence, an affinity measurement, and an expression measurement respectively.

A machine learning model is trained to serve as a property oracle for both an affinity property and an expression property. The model used in this process, dubbed “Omniprot,” is an adaptation of the BERT masked language model described in further detail in Devlin, Jacob, et al. “Bert: Pre-training of deep bidirectional transformers for language understanding.” arXiv preprint arXiv: 1810.04805 (2018) (hereinafter “Devlin”), which is hereby incorporated by reference in its entirety. In principal, however, any model that can be pretrained in an unsupervised manner can be used. Omniprot is a deep transformer model which is trained by learning to reconstruct masked portions of a protein sequence, s_{masked}, which has a random 15% of its amino acids masked. The training task can be stated as,

$$\tilde{θ} = {arg max}_{θ} p ( {s}_{masked} \vert {s}_{unmasked} , θ )$$

where θ is a vector of Omniprot's parameters and \tilde{θ} denotes the network parameters which are an optima solving the unsupervised pretraining problem. In other words, Omniprot is evaluated on how accurately it can guess the masked amino acids. This training objective teaches Omniprot to model the statistical regularities in the underlying protein sequence space. In this case, Omniprot trains on the unsupervised antibody sequence dataset, [symbol] , as a form of transfer learning to improve model performance on smaller downstream supervised tasks. Devlin further discloses aspects of unsupervised pretraining of BERT-like models and their architecture.

Once a set of model parameters, \tilde{θ}, is obtained from the pretraining objective, a much smaller supervised set of data can be used in finetuning the model. In this example embodiment, finetuning means, after using the pretraining parameters to initialize the model at the start of training, solving a new optimization problem to adapt the model to a new task using a smaller data set. To this end, the finetuning optimization problem can be represented as

$$\hat{θ} = {arg max}_{θ} {\sum }_{y ∈ ( a , e )} {w}_{y} p ( y \vert s , θ )$$

where w_{y}∈ [symbol] is a hyperparameter that weights each sequence property of interest, and \hat{θ} is the optimal set of network weights given the training dataset [symbol] . The optimal parameters along with the model architecture constitute an oracle which can now be used to do downstream optimization of antibody affinity.

Once a model has been trained, the model can be used to find antibody sequences with improved affinity. Such a step is referred to as sequence maturation. In an example embodiment, a finetuned Omniprot model can predict affinity and expression of candidate sequences and search over a valid domain of possible antibody sequences. The optimization problem can be expressed as

$$\hat{s} = {arg max}_{s} {f}_{\hat{θ}} ( s )$$

where f_{\hat{θ}} is the finetuned Omniprot model's map from sequence to affinity. This optimization problem can be solved naively in many ways by applying different optimization methods. However, an important consideration is restricting the domain (e.g., protein sequences) to a set of valid antibody sequences. To solve this problem while taking into account the desired sequence constraints, an optimization procedure called Computational Directed Evolution (hereinafter “CDE”) can be applied. CDE is a Markov Chain Monte Carlo [MCMC] technique applied to protein optimization. The CDE's MCMC process uses a proposal distribution specified by the marginal probabilities of a mutation in an MSA of the training set. The process demonstrates how each mutation improves the predicted affinity by the model in expectation. When iterated, additional mutations are accumulated in the original sequences that improve predicted binding affinity, ultimately resulting in a strong candidate for testing in the lab. More specifically, CDE is an implementation of the Metropolis-Hastings method. In this example embodiment, CDE is a biased random walk through the valid protein sequence space where the highest affinity antibody found is returned at the chosen end of the walk.

In order to implement a Metropolis-Hastings method, a collection of objects are used as inputs:
a) an initial starting antibody sequence so E
b) a proposal distribution p(s_{t+1}|s_t), and
c) an acceptance criterion g: [symbol] *× [symbol] *→[0,1],
   where [symbol] is the set of all antibody sequences in the training set of the Omniprot finetuning process, and [symbol] * is the set of all sequences that can be drawn from the marginal distribution over amino acids in the aligned training set. As such, acceptance criterion g is a map from a pair of sequences (e.g., an antibody sequence and a candidate next sequence) to the unit interval indicating the probability of accepting the candidate sequence as the next step in the random walk. In this example embodiment of the method, the acceptance criterion is defined as:

$$g ( s , {s}_{c} ) = min ( {e}^{α ( {f}_{\hat{θ}} ( s ) - {f}_{\hat{θ}} ( {s}_{c} ) )} , 1 )$$

where s and s_{c} are the current sequence and candidate sequence respectively, and α is a positive constant. In principal, many such acceptance criterion can be used and be effective, however in practice the criterion above is found to be effective.

Given the above, the CDE method as follows (noting that this is an implementation of Metropolis-Hastings):
a) Start with an initial antibody sequence: s←s₀
b) Initialize the current best sequence: s_{b}←s₀
c) Draw a new candidate sequence from the proposal distribution: s_{c}˜p( [symbol] |s)
d) With probability: g(s,s_{c}), set s←s_{c}
e) If f_{\hat{θ}}(s_{b})<f_{\hat{θ}}(s),: s_{b}←s
f) Return to step (c) until n>max_iterations

A designer may want to optimize multiple properties of the antibody sequence. To do that, a model can be employed to estimate each property of the antibody sequence, and an objective function combines the weighted results of the respective models using hyperparameters. The hyperparameters and use thereof are described below.

Consider the scenario where two objectives are being optimized. A person having ordinary skill in the art can recognize that any two properties can be analyzed. For this example, however, binding affinity and antibody solubility are being used for illustration. For example, a first model determines the binding affinity of the sequence to our target (m₁(s)). The second model determines the antibody solubility (m₂(s)). Both models take input a sequence (s) and return a scalar quantity representing the property being measured. A joint objective function (e.g., an objective function) can combine the two scalar outputs as follows:

objective(s):=(1−α)m₁(s)+αm₂(s)

where α is on the closed interval from 0 to 1. Determining this a hyperparameter is part of the practice of finding a good objective function, and can require testing. In general, the objective can be expressed as a weighted sum of the set of models. Optimizing multiple models involves determining how to weight each model when combining them to get a desired antibody sequence. Such an optimization can be performed manually, as the designer of the antibody may prioritize certain properties over others. The hyperparameter can represent one or more of an importance of the property to manufacturing (e.g., manufacturability), an expression of a protein, an immunogenicity in a patient, developability, an interaction with other models, orthogonality with other models, and an empirical derivation by tuning the generation process. Manufacturability is a factor based on the ease or difficulty of creating a protein sequence (e.g., medicine) using standard biochemical techniques. Manufacturability factors include: how easy the protein is to express, how likely the protein is to aggregate, how stable the protein is, etc.. These concerns are all related to the cost and feasibility of production. Developability is a factor based on attributes relating to clinical success of a protein sequence (e.g., medicine). The developability factors include how easy the protein is to express, how likely the protein is to aggregate, how stable the protein is, specificity to a target, etc.

In general, for n models, a multimodel type of objective function is represented as:

$$objective ( s ) := {\sum }_{i = 1}^{n} {w}_{i} {m}_{i} ( s )$$

where the weights wᵢ take values between 0 and 1, and the total of all weights wᵢ sum to 1. When only one property is being optimized, n=1 and wᵢ=1. However, a designer configures the weights for multimodel objective functions.

FIG. 1B is a flow diagram 150 illustrating an example embodiment of a process employed by the present disclosure. The process begins by selecting an initial antibody s₀ (152). The initial antibody so is the antibody to be improved upon by the machine learning process. To initialize, the method sets the best found antibody s_{b} to be s₀ for the first pass. Then, the method draws a new sequence s_{c} from a proposal distribution as defined above, s_{c}˜p( [symbol] |s) (156). In other words, the proposal distribution selects a new antibody that is a single sequence from the current sequence, so. Given the proposal distribution s_{c}˜p( [symbol] |s), a new sequence is proposed by selecting a position to mutate using a uniform distribution over positions. Once a position is selected, there are several ways in which the particular point mutation is selected. These methods include the following:
a) Uniformly randomly select a new amino acid (from the canonical set of 20) at the position of interest,
b) Select a new amino acid at the position of interest by using the first order distribution induced by a multiple sequence alignment of the antibodies in the training set, or
c) Select a new amino acid at the position of interest by sampling over the zero order distribution of amino acids found in the antibody training set.

A first order distribution induced by multiple sequence alignment is the empirical distribution of possible amino acids seen at a particular position in the training set. A zero order distribution is the empirical distribution of all amino acids regardless of position. A zero order distribution is similar to taking every residue from each protein in the training set, putting them into a bag and sampling from that bag without replacement. The zero order distribution does not preserve position, whereas the first order distribution considers the distribution at each position in the protein. The process described above induces a distribution of possible sequences, and a conditional distribution given a starting sequence.

If the probability g(s,s_{c}) is over a configurable threshold, s is set to be s_{c} (158). The configurable threshold can be set by a designing user in one embodiment.

In another embodiment, the configurable threshold can be set automatically by at least on factor. Whether automatic or manual, the configurable threshold can be set considering the following factors: the rate of acceptance of proposals, and the rate of mixing of the MCMC procedure. If the threshold is set too low, the acceptance rate will be high, but mixing will be low, and convergence will be slow. If the threshold is set too high, mixing will be high, but the acceptance rate will be low, so again convergence will be slow. Therefore, to maximize the performance of the algorithm, an intermediate value that reaches a balance between the proposal acceptance rate and the mixing rate is ideal. As described above, g(s,s_{c}) represents a map from a pair of sequences (e.g., an antibody sequence and a candidate next sequence) to the unit interval indicating the probability of accepting the candidate sequence as the next step in the random walk.

The method then determines whether the property value found from f_{\hat{θ}}(s_{b}) is less than the property value of f_{\hat{θ}}(s) (160). In other words, the method compares the affinity values of s_{b} and s, however, a person of ordinary skill in the art can recognize that other property values can be evaluated at this step other than affinity. If the evaluated property of s_{b} is less than s, s is set to be s_{b} (e.g., the sequence being considered is evaluated as the best sequence so far) (162). Then, the method checks whether it has iterated enough times or other metric indicating the method is complete (164). If so, the method outputs s_{b} as the output (166). If not, the method draws another sequence s_{c} from the proposal distribution (156). If the evaluated property of s_{b} is greater than or equal to s, the method checks whether it is complete (164) and continues as described above from there.

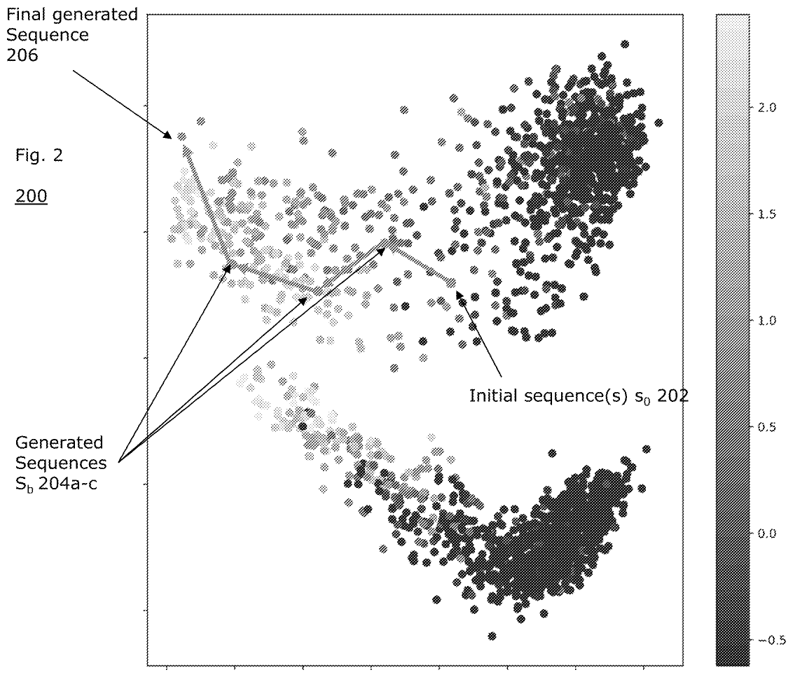

FIG. 2 is a graph 200 illustrating a random walk sequence in an antibody sequence space for discovering antibody sequences using Computational Directed Evolution. Using CDE with the finetuned model optimizes antibody affinity. Seed antibody sequences so 202 are the one or more initial antibody sequences. The best sequence of those sequences, s_{b} 204 is chosen to be initialized, where the best is the one with the highest affinity and expression properties. Then, a new candidate sequence 206 is drawn from a proposal distribution. The set of sequences s is then set to be s_{c}. When the property value results of the finetuned model for s_{b} are less than for s, s_{b} is set to be s. Then, the process repeats until a suitable antibody is found.

The result of the method described herein provides an improved antibody sequence s_{b}. This walk is iterated to produce as many sequences as needed for testing. In embodiments, the walk can be random, guided, or a hybrid approach. These sequences are then assayed for affinity to the antigen and expression. The data can then be fed back into the finetuning process and can be repeated as many times as desired until clinically relevant antibody sequences are created. Using the methods described herein, antibody sequences can be generating having improved Fluorescein antibody affinity with an order of magnitude over the highest affinity antibody seen in the dataset. In this case, pretraining and finetuning used the datasets described above, but other datasets can be used.

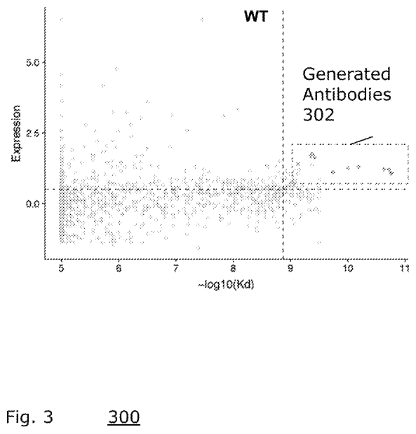

FIG. 3 is a graph 300 illustrating the improvements of the generated antibody sequences 302 compared to existing datasets. The graph illustrates generated fluorescein antibody sequences using de novo antibody affinity maturation. Using the de novo antibody affinity maturation process, sub-100 pM affinity fluorescein antibody sequences are generated. In the graph 300, the antibody sequences are plotted with the property of affinity (x-axis) versus expression (y-axis). The upper right quadrant includes the generated antibody sequences 302 having the highest of both properties. A person of ordinary skill in the art can recognize that these generated antibody sequences 302, illustrated in red, have the properties desired by the process.

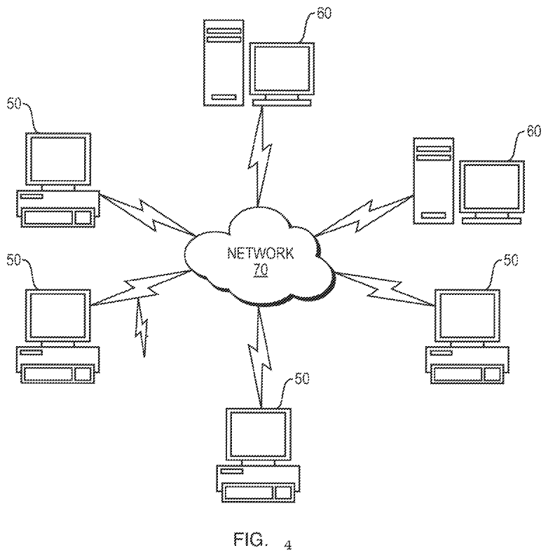

FIG. 4 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

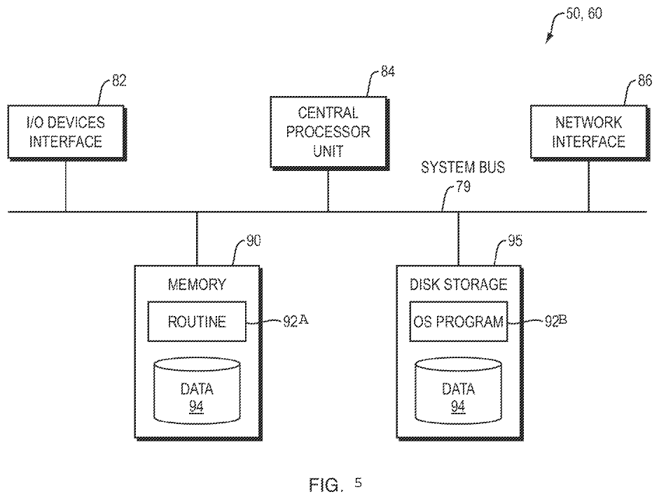

FIG. 5 is a diagram of an example internal structure of a computer (e.g., client processor/device 50 or server computers 60) in the computer system of FIG. 4. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., machine learning model module and fine-tuned machine learning model module code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method for determining an antibody sequence having an improved property, the method comprising:
generating a score for each of a plurality of machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence, resulting in a respective score for each machine learning model of the plurality of machine learning models indicating a contribution to predicting the property corresponding to the respective machine learning model; and
generating an antibody sequence using the plurality of machine learning models by weighting outputs of each machine learning model according to each score generated and combining the weighted outputs into a weighted sum.

2. The method of claim 1, wherein generating the antibody sequence further includes:
selecting an antibody sequence from a proposal distribution based on the plurality of machine learning models; and
determining whether the antibody sequence selected has a probability of acceptance over a particular threshold, and if so, analyzing the antibody sequence, and if not, selecting a next antibody sequence from the proposal distribution.

3. The method of claim 1 or 2, wherein generating the antibody sequence further includes:
comparing a first property value, determined by a function of the plurality of machine learning models, of an antibody sequence selected from a proposal distribution to a second property value, determined by the plurality of machine learning models, of an antibody sequence having a best property value in a current search;
if the first property value is greater than the second property value, replacing the antibody sequence having the best property value with the antibody sequence selected from the proposal distribution.

4. The method of any one of the preceding claims, wherein generating the fine-tuned machine learning model further includes:
weighting each sequence property of the second plurality of antibody sequences;
determining optimum model parameters to generate the second plurality of antibody sequences using the machine learning model; and
applying the optimum model parameters to the machine learning model, the resulting model having the optimum model parameters applied being the fine-tuned machine learning model.

5. The method of any one of the preceding claims, wherein the corresponding property is at least one of affinity, expression, protein aggregation, proteolytic stability, expression, and off target effects.

6. The method of any one of the preceding claims, further comprising:
selecting an antibody sequence candidate from a proposal distribution based on the plurality of machine learning models, the antibody sequence being within a defined acceptance criterion; and
if the property of the antibody sequence candidate is greater than a best found antibody sequence, replacing the best found antibody sequence with the antibody sequence candidate, or otherwise, disregarding the antibody sequence candidate.

7. The method of any of the preceding claims, the method further comprising:
producing an antibody having the antibody sequence generated.

8. The method of any of the preceding claims, the method further comprising:
providing a manufactured antibody having the antibody sequence generated; and
assaying the antibody for the property.

9. The method of any of the preceding claims, further comprising:
training one or more machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence.

10. The method of claim 9, wherein training the one or more machine learning models further includes:
providing a set of amino acid sequences labeled with at least one property;
masking a portion of the set of amino acid sequences to provide a masked set of amino acid sequences, wherein the remainder of the set of amino acid sequences is an unmasked set of amino acid sequences;
training the one or more machine learning models to estimate each amino acid sequence of the masked set based on (1) the at least one property labeling each masked amino acid sequence and (2) the unmasked set of amino acid sequences and the labeled properties of each unmasked amino acid sequence.

11. The method of any of the preceding claims, wherein generating the antibody sequence is performed by employing MCMC sampling.

12. The method of any of the preceding claims, wherein the plurality of antibody sequences is related to an antigen of interest.

13. The method of any of the preceding claims, wherein the contribution is to predict at least one of the following properties to be improved:
an importance of the property to manufacturing of the antibody sequence,
an immunogenicity of the antibody in a patient,
expression level of the antibody,
developability,
an interaction with other models,
orthogonality with other models, and
an empirical derivation by tuning the generation process.

14. A system for determining an antibody sequence having an improved property, the method comprising:
a processor; and
a memory with computer code instructions stored thereon, the processor and the memory, with the computer code instructions, being configured to cause the system to:
generate a score for each of a plurality of machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence resulting in a respective score for each machine learning model of the plurality of machine learning models indicating a contribution to predicting the property corresponding to the respective machine learning model; and
generate an antibody sequence using the plurality of machine learning models by weighting outputs of each machine learning model according to each score generated and combining the weighted outputs into a weighted sum.

15. The system of claim 14, wherein generating the antibody sequence further includes:
selecting an antibody sequence from a proposal distribution based on the fine-tuned machine learning model; and
determining whether the antibody sequence selected has a probability of acceptance over a particular threshold, and if so, analyzing the antibody sequence, and if not, selecting a next antibody sequence from the proposal distribution.

16. The system of any one of claims 14-15, wherein generating the antibody sequence further includes:
comparing a first property value, determined by a function of the fine-tuned machine learning model, of an antibody sequence selected from a proposal distribution to a second property value, determined by the fine-tuned machine learning model, of an antibody sequence having a best property value in a current search;
if the first property value is greater than the second property value, replacing the antibody sequence having the best property value with the antibody sequence selected from the proposal distribution.

17. The system of any one of claims 14-16, wherein generating the fine-tuned machine learning model further includes:
weighting each sequence property of the second plurality of antibody sequences;
determining optimum model parameters to generate the second plurality of antibody sequences using the machine learning model; and
applying the optimum model parameters to the machine learning model, the resulting model having the optimum model parameters applied being the fine-tuned machine learning model.

18. The system of any one of claims 14-17, wherein the corresponding property is at least one of affinity and expression.

19. The system of any one of claims 14-18, wherein the processor is further configured to:
select an antibody sequence candidate from a proposal distribution based on the finely-tuned machine learning model, the antibody sequence being within a defined acceptance criterion; and
if the property of the antibody sequence candidate is greater than a best found antibody sequence, replace the best found antibody sequence with the antibody sequence candidate, or otherwise, disregarding the antibody sequence candidate.

20. The system of any one of claims 14-19, wherein the processor is further configured to:
train one or more machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence.

21. The system of claim 20, wherein training the machine learning model further includes:
providing a set of amino acid sequences labeled with at least one property;
masking a portion of the set of amino acid sequences to provide a masked set of amino acid sequences, wherein the remainder of the set of amino acid sequences is an unmasked set of amino acid sequences;
training the machine learning model to estimate each of the masked set of amino acid sequences based on (1) the at least one property labeling each masked amino acid sequence and (2) the unmasked set of amino acid sequences and the labeled properties of each unmasked amino acid sequence.

22. The system of any of claims 14-21, wherein generating the antibody sequence is performed by employing MCMC sampling.

23. The system of claim 14-22, wherein the plurality of antibody sequences is related to an antigen of interest.

24. The system of claim 14-23, wherein the contribution is to predicting at least one of the following properties to be improved:
an importance of the property to manufacturing of the antibody sequence,
an expression of the antibody sequence,
an immunogenicity of the antibody in a patient,
expression level of the antibody,
developability,
an interaction with other models,
orthogonality with other models, and
an empirical derivation by tuning the generation process.

25. A method of antibody maturation comprising:
providing a first antibody sequence to the system of any one of claims 9-16; and
obtaining, from the system, the generated antibody sequence.

26. An isolated antibody produced by the method of claim 25.

27. The isolated antibody of claim 26, wherein the isolated antibody is recombinantly produced.

28. The isolated antibody of claims 26-27, wherein the isolated antibody is chemically synthesized.

29. A method of antibody maturation comprising:
providing a first antibody sequence to the method of any one of claims 1-9; and
obtaining, from the system, the generated antibody sequence.

30. An isolated antibody produced by the method of claim 29.

31. The isolated antibody of claim 30, wherein the isolated antibody is recombinantly produced.

32. The isolated antibody of claims 30-31, wherein the isolated antibody is chemically synthesized.

33. A method for determining an antibody sequence having an improved property, the method comprising:
generating a score for each of a plurality of fine-tuned machine learning models trained based on a corresponding initial plurality of antibody sequences, each antibody sequence of the initial plurality of antibody sequences being labeled with a corresponding property, each fine-tune machine learning model further generated by training each machine learning model with a secondary plurality of antibody sequences, each antibody sequence of the secondary plurality of antibody sequences being labeled with a corresponding property, the secondary plurality of antibody sequences related to an antigen of interest; and
generating an antibody sequence based an objective function using the plurality of fine-tuned machine learning models weighted by corresponding hyperparameters.

34. A method for determining an antibody sequence having an improved property, the method comprising:
training a machine learning model based on a first plurality of antibody sequences, each antibody sequence of the first plurality of antibody sequences being labeled with a corresponding property;
generating a fine-tuned machine learning model by training the machine learning model based on a second plurality of antibody sequences, each antibody sequence of the second plurality of antibody sequences being labeled with a corresponding property, the second plurality of antibody sequences related to an antigen of interest; and
generating an antibody sequence based on the fine-tuned machine learning model.

35. A method for determining an antibody sequence having an improved property, the method comprising:
providing a score for each of a plurality of machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence, each a respective score for each machine learning model of the plurality of machine learning models indicating a contribution to predicting the property corresponding to the respective machine learning model; and
generating an antibody sequence using the plurality of machine learning models by weighting outputs of each machine learning model according to each score provided and combining the weighted outputs into a weighted sum.

36. A method for determining an antibody sequence having an improved property, the method comprising:
generating an antibody sequence using a plurality of machine learning models by:
weighting outputs of each machine learning model according to a score for each of the plurality of machine learning models, each machine learning model trained based on a respective plurality of antibody sequences, each antibody sequence of the respective plurality of antibody sequences labeled with a property corresponding to the plurality of antibody sequences and a value of the property corresponding to the respective antibody sequence, each respective score for each machine learning model of the plurality of machine learning models indicating a contribution to predicting the property corresponding to the respective machine learning model, and
combining the weighted outputs into a weighted sum.
